(19) United States
(12) Patent Application Publication
(10) Pub. No.: US 2002/0110928 A1
(43) Pub. Date: Aug. 15, 2002

(54) Evaluation apparatus for biological samples, evaluation method for the same, and storage medium storing computer programs for executing the same evaluation method

(76) Inventor: Kanji Yahiro, Oonojo-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/042,312

(22) Filed: Jan. 11, 2002

(30) Foreign Application Priority Data
Jan. 12, 2001 (JP) 2001-4685

(51) Int. Cl. G01N 33/543; G06F 19/00; G01N 33/48; G01N 33/50; G06K 9/00

(52) U.S. Cl. 436/518; 702/19; 382/128

(57) ABSTRACT

An evaluation apparatus of a biological sample for acquiring numerical data showing the state of biological samples from the image data obtained by taking an image of the biological samples, an evaluation method, and a storage medium storing a program for executing such evaluation method are disclosed. A condition pass/fail determining unit determines if the measuring area being set as numerical data acquiring area in the image to be evaluated conforms to the predetermined condition as the condition for acquiring numerical data. A digitization unit obtains the numerical data from the image of which measuring area is judged to be conforming to the predetermined condition. A measuring area changing unit changes the measuring area. The evaluation apparatus, evaluation method, and storage medium are capable of acquiring highly objective numerical data efficiently only from appropriate objects.

### FIELD OF THE INVENTION

The present invention relates to an evaluation apparatus biological samples for acquiring numerical data from the image data obtained by taking images of biological samples such as cells of animals and plants, an evaluation method, and a storage medium storing computer programs for executing the same evaluation method.

### BACKGROUND OF THE INVENTION

In various tests in biochemical fields, biological samples such as cells of animals and plants or microorganisms are often cultured in various conditions and observed. In such observation, numerical data for evaluation of specific items are obtained from the images obtained by microscopic observation. When observing the nerve cell, for example, the degree of growth of a neurite extending from the nerve cell is digitized to obtain numerical data.

Hitherto, such evaluation of biological samples depended on manual operations.

In the evaluation job, the person in charge of experiment visually observes the nerve cells in the viewing field of the microscope, notices the site of observation, counts the measuring positions, acquires the size by visually comparing with the scale, and digitizes and records the data.

Such observation work itself takes much time and labor. Therefore, this observation causes the efficiency of the entire experiment to lower and demands an excessive load for the person in charge of experiment.

To digitize the observation result, generally, proper samples are extracted from all materials, and numerical data is obtained by measuring the size or counting the number in the acquired measuring area. Depending on the measuring area, however, adequate results suited to the purpose of measurement may not be always obteained.

For example, when measuring the degree of growth of a neurite of a nerve cell as mentioned above, if a range of an excessively concentrated proliferation of cells is determined as measuring area, grown axons are concealed by the proliferated cells and are not observed. As a result, data showing correct degree of growth may not be obtained. Hitherto, including the selection of such measuring area, the observation work depended on the know-how and experience of the workers. Therefore, the precision and reliability of data varied significantly depending on the experience and skill of the individual workers. It was hence difficult to acquire highly objective numerical data efficiently in the conventional evaluation of biological samples.

### SUMMARY OF THE INVENTION

It is hence an object of the invention to present an evaluation apparatus of biological samples capable of acquiring highly objective numerical data efficiently from images of biological samples, an evaluation method, and a storage medium storing programs for executing the evaluation method by a computer.

The evaluation apparatus of biological samples of the invention acquires numerical data showing the state of biological samples from the image data obtained by taking an image of the biological samples. In this evaluation apparatus, a condition pass/fail determining unit determines if the measuring area being set as numerical data acquiring area in the image to be evaluated conforms to the predetermined condition as the condition for acquiring numerical data. A digitization unit acquires the numerical data from the image of which measuring area is judged to conform to the predetermined condition. A measuring area changing unit changes the measuring area.

The evaluation method of biological samples of the invention is a method of acquiring numerical data from the image data obtained by taking the biological samples. This evaluation method comprises the following steps.

(a) Setting the condition for acquiring numerical data from the measuring area being set as numerical data acquiring area in the image to be evaluated.

(b) Judging whether the measuring area conforms to the condition or not when acquiring the numerical data.

(c) Acquiring the numerical data from the measuring area when judged to conform to the condition.

(d) Changing the measuring area when judged not to conform.

The storage medium of the invention is a storage medium storing a computer program for executing an evaluation method of biological samples for acquiring numerical data from the image data obtained by taking the image of the state of biological samples. A computer can read this storage medium. This computer program makes the computer execute this evaluation method. The evaluation method stored in the computer program comprises the same steps as the evaluation method of biological samples.

According to the invention, the condition for obtaining numerical data appropriately from the measuring area determined as the numerical data acquiring area in the image to be evaluated is set as a predetermined condition. When obtaining numerical data, it is judged if the measuring area conforms to the condition or not by the condition pass/fail determining unit. Numerical data is acquired from the measuring area judged to conform to the condition. Thus, highly objective numerical data is acquired only from the appropriate object.

### BRIEF DESCRIPTION OF THE DRAWINGS

FIG. 1 is a perspective view of an evaluation apparatus of biological samples in an embodiment of the invention.

FIG. 2 is a block diagram showing a configuration of the evaluation apparatus of biological samples in the embodiment of the invention.

FIG. 3 is a function block diagram showing functions of the evaluation apparatus of biological samples in the embodiment of the invention.

FIG. 4 is a process flowchart of basic operation program of the evaluation apparatus of biological samples in the embodiment of the invention.

FIG. 5 is a process flowchart of basic operation program of the evaluation apparatus of biological samples in the embodiment of the invention.

FIG. 6A, B, C show images of biological samples to be measured by the evaluation apparatus of biological samples in the embodiment of the invention.

### DESCRIPTION OF THE PREFERRED EMBODIMENT

A preferred embodiment of the invention is explained below referring to the accompanying drawings.

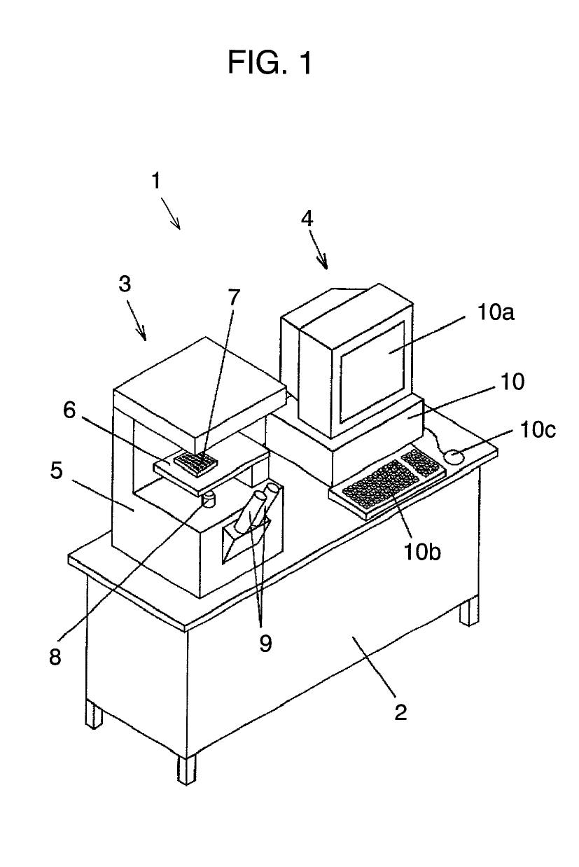

FIG. 1 is a perspective view of an evaluation apparatus of biological samples in an embodiment of the invention.

This evaluation apparatus of biological samples is used for acquiring numerical data for evaluating the state of biological samples such as nerve cells contained in a container, that is, digitized data of the state of nerve cells.

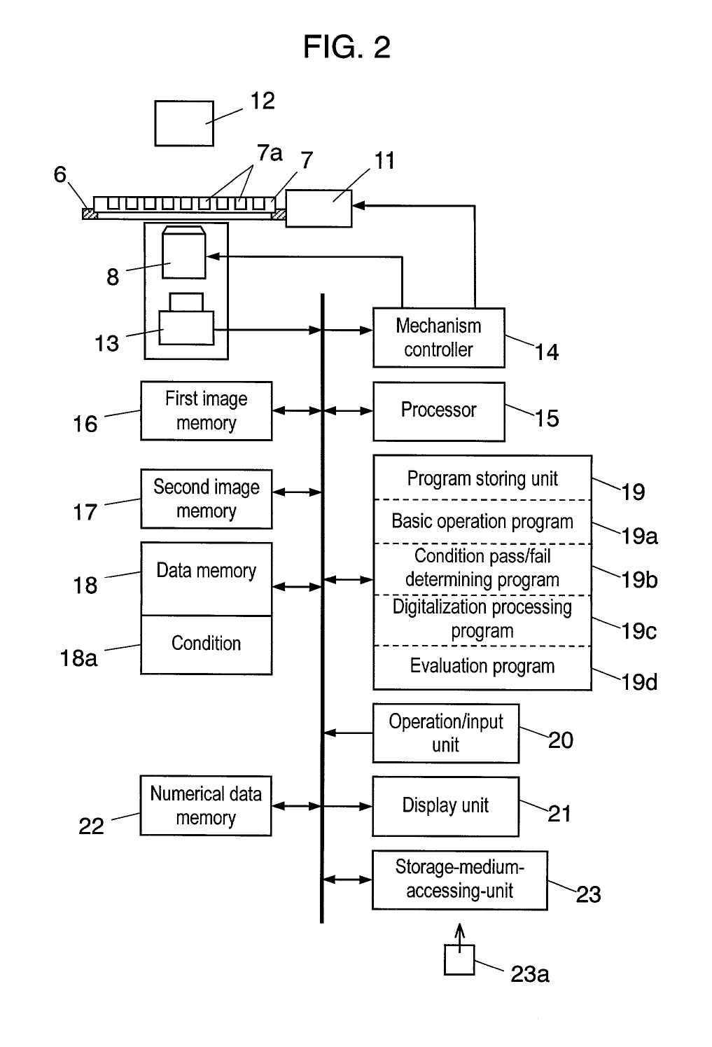

In FIG. 1, an evaluation apparatus of biological samples 1 is constituted by putting a microscopic observation unit 3 and an operation/calculation unit 4 parallel on a stand 2. The microscopic observation unit 3 includes a camera incorporated in a casing 5, and a sample table 6 for holding a micro plate 7 which is a container of biological samples such as cells of animals and plants. The camera takes microscopic images of biological samples contained in the micro plate 7 on the sample table 6 through an optical system 8. The camera is explained in detail later by referring to FIG. 2.

The microscopic observation unit 3 also includes an ocular lens 9. Therefore, the biological sample in the micro plate 7 can be visually observed through the ocular lens 9.

The operation/calculation unit 4 includes a personal computer 10. The personal computer 10 displays the image taken by the microscopic observation unit 3 on its monitor 10a. In the personal computer 10, while displaying the input screen and operation screen on the monitor 10a, various data and operation commands are entered by a keyboard 10b and a mouse 10c.

Referring next to FIG. 2, the configuration of the microscopic observation unit 3 and control system is explained.

FIG. 2 is a block diagram showing a configuration of the evaluation apparatus of biological samples in the embodiment of the invention.

In FIG. 2, the micro plate 7 has multiple wells 7a as containers of biological samples, disposed in a lattice form. The micro plate 7 is held on the sample table 6. The sample table 6 is moved horizontally in the XY direction by means of a table moving mechanism 11. Below the sample table 6, the optical system 8 and camera 13 are disposed. The camera 13 takes an image of the biological sample in the well 7a illuminated by an illumination unit 12 disposed above. Therefore, the camera 13 functions as an imaging unit for taking the microscopic image of biological sample in the container.

A mechanism controller 14 controls the optical system 8 and table moving mechanism 11.

The table moving mechanism 11 functions as a moving unit for moving the micro plate 7 relatively to the camera 13. The table moving mechanism 11 can move the imaging field of the camera 13 to a desired position of a desired well 7a. Therefore, the table moving mechanism 11 can move and change the measuring area determined as an object of numerical data acquisition in the image obtained by taking the image of the biological sample in the well 7a, to a desired position.

A processor 15 is a CPU, which calculates and processes by executing various programs stored in a program storing unit 19 according to the data stored in a data memory 18.

A first image memory 16 stores the image data taken by the camera 13.

A second image memory 17 stores the image processed as a result of image processing the image data being read out from the first image memory 16.

The data memory 18 stores a condition 18a and other various data.

The condition 18a is a condition predetermined as the condition for acquiring numerical data appropriately from the image to be evaluated. The condition 18a specifies, for example, the number of cells in the measuring area being set as the sample area for obtaining numerical data in the image, or the adequate range of the sum of areas.

The program storing unit 19 stores a basic operation program 19a, a condition pass/fail determining program 19b, a digitization processing program 19c, and an evaluation program 19d.

The basic operation program 19a is a processing program of a basic operation by the evaluation apparatus of biological samples 1.

The condition pass/fail determining program 19b judges whether or not the image from which numerical data is to be acquired conforms to the predetermined condition as the condition for adequately obtaining the numerical data from the image to be evaluated.

In this embodiment, as mentioned above, the number of cells in the measuring area or the surface area being in a predetermined range is set as the condition to be satisfied.

The digitization processing program 19c, is a program for digitization-processing to acquire desired numerical data from the image to be evaluated.

In this embodiment, the length or area of linear structure is obtained as numerical data as the numerical index expressing the degree of growth of linear structure, for example, neurite extending from soma, for example, nerve cell in the measuring area.

The evaluation program 19d is a program for executing various processes, such as processing of the obtained numerical data in a specified graph format to be evaluated visually, and comparison of the numerical data with preset reference value to display the result of comparison graphically.

An operation/input unit 20 includes the keyboard 10b and mouse 10c of the personal computer 10, and input operation is made on the operation screen.

A display unit 21 is the monitor 10a of the personal computer 10, and displays the taken images and operation screen.

A numerical data memory 22 stores various numerical data obtained by digitizing the taken images.

A storage medium accessing unit 23 includes a drive device of an external storage medium 23a such as a CD-ROM and floppy disk. The external storage medium 23a stores the basic operation program 19a, condition pass/fail determining program 19b, digitization processing program 19c, and evaluation program 19d. The accessing unit 23 reads these programs from the external storage medium 23a. Accordingly, an ordinary personal computer having functions of input, memory, calculation and display can be used as the processing unit of the evaluation apparatus of the biological samples.

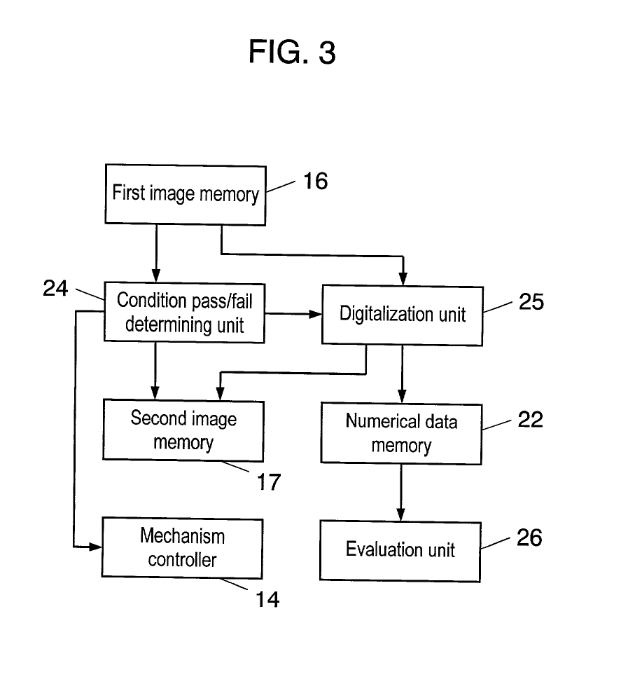

Referring now to FIG. 3, the processing function of the evaluation apparatus of the biological samples is explained.

FIG. 3 is a function block diagram showing functions of the evaluation apparatus of biological samples in the embodiment of the invention.

In FIG. 3, a condition pass/fail determining unit 24, a digitization unit 25, and an evaluation unit 26 have functions to be realized by executing the condition pass/fail determining program 19b, digitization processing program 19c, and evaluation program 19dwith the processor 15.

When acquiring the numerical data, first, the taken image stored in the first image memory 16 is read into the condition pass/fail determining unit 24. Herein, the condition pass/fail determination is made. The determining unit 24 judges if the image to be digitized conforms to the predetermined condition as the condition for adequately acquiring the numerical data from the image to be evaluated.

If the image is judged to conform to the predetermined condition, it is to be digitized in the digitization unit 25.

The numerical data acquired in the digitizing process is stored in the numerical data memory 22. The stored numerical data is read into the evaluation unit 26, and various evaluations are processed according to these numerical data.

The image digitized by the digitization unit 25 is stored in the second memory 17.

If the measuring area is judged not to conform to the predetermined condition in the condition pass/fail determining unit 24, the mechanism controller 14 controls the table moving mechanism 11, and moves the micro plate 7 relatively to the camera 13. Thus, the imaging field is moved to a new measuring area. As a result, the new measuring area is set in the imaging field, and the image of this measuring area is taken. In other words, the mechanism controller 14 functions as the measuring area changing unit for changing the measuring area being set as the numerical data acquisition area.

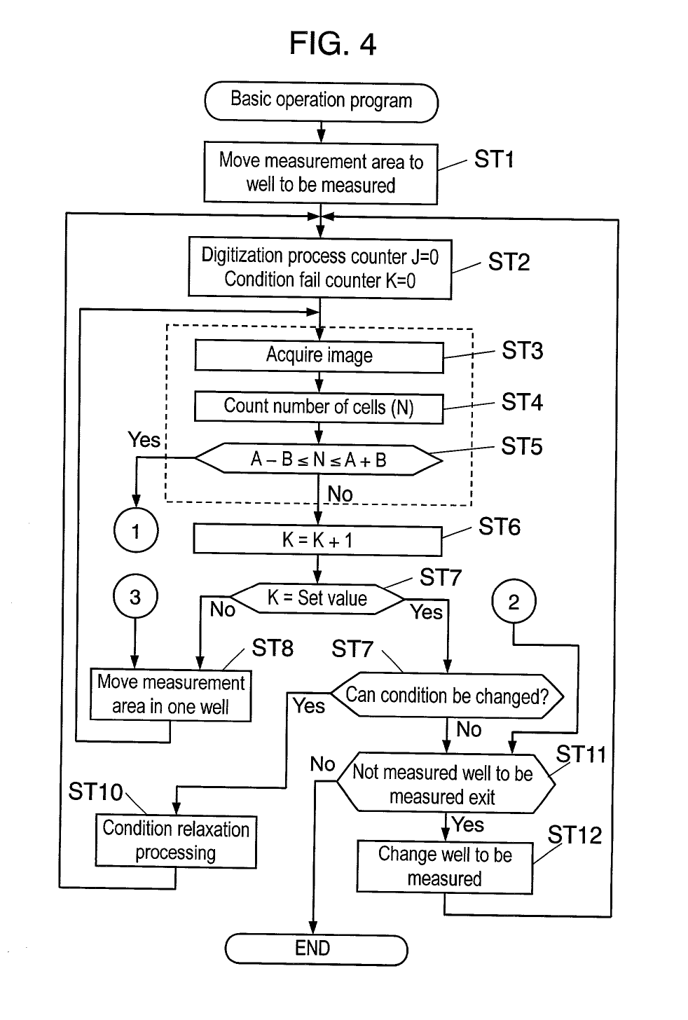

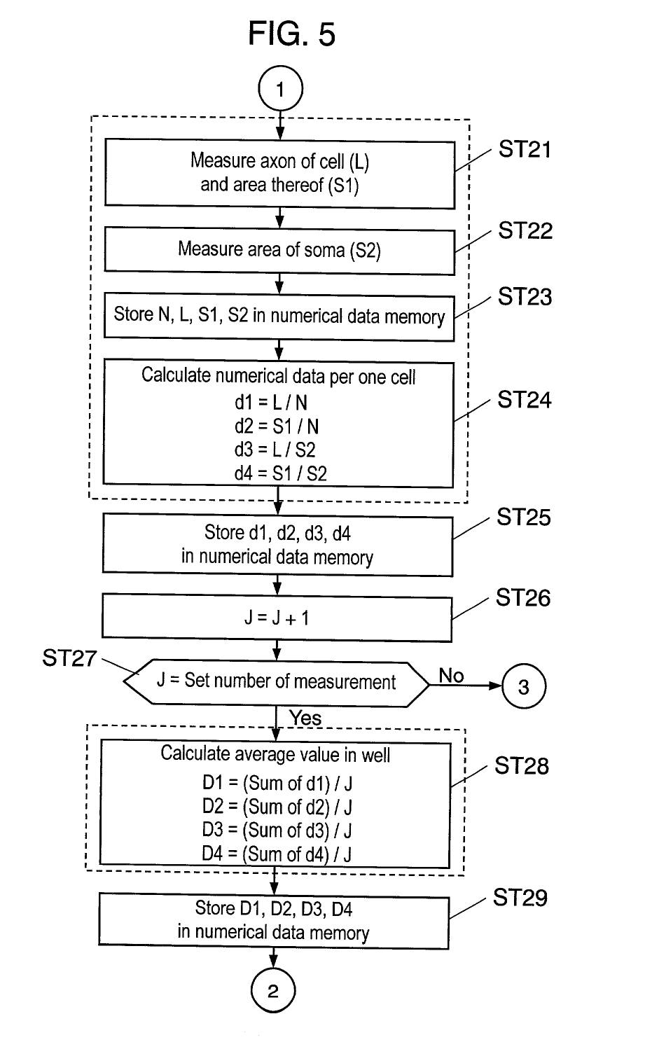

Referring next to FIG. 4 and FIG. 5, the processing flow by the basic operation program of the evaluation apparatus of biological samples is explained.

FIG. 4 and FIG. 5 are process flowcharts of basic operation program of the evaluation apparatus of biological samples in the embodiment of the invention.

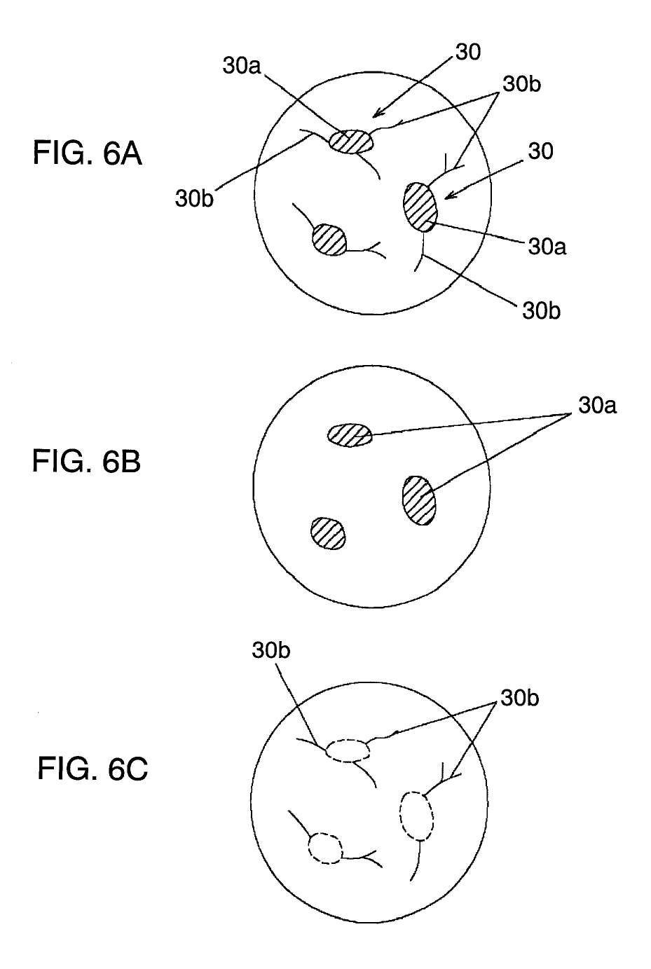

FIG. 6 shows images of biological samples to be measured by the evaluation apparatus of biological samples in the embodiment of the invention.

In FIG. 4, when the micro plate 7 containing biological samples to be evaluated is put on the sample table 6, and evaluation process is started, the imaging field of the camera 13 is moved to the measuring area of the well 7a to be measured (ST1).

Then, the digitization process counter J and condition fail counter K are initialized to zero (ST2).

The condition is determined (ST3 to ST5).

The camera 13 acquires the image (see FIG. 6A) to be determined (ST3).

The number of cells N is counted as the soma numerical index in the measuring area (ST4).

This count is based on the image obtained by processing the image shown in FIG. 6A. Herein, the linear structures 30b is deleted from the image showing the soma 30 in the image, and the image leaving only the main bodies 30a of the soma 30 as shown in FIG. 6B is obtained. On this processed image, by counting the number of main bodies 30a, the number of cells N is determined.

Next, it is judged whether the determined number of cells N conforms to the predetermined condition. Herein, it is judged whether the number of cells N is within a range between (A−B) and (A+B) or not (ST5).

Value A is the reference of the number of cells N as the criterion. Value B is the numerical value showing the allowable range width. When the number of cells N conforms to this condition, the data is digitized as explained below (ST24).

If not conforming, the following process is done.

First, adding 1 to the value of condition fail counter K, K=K+1 is set (ST6).

It is judged whether this counter value K have reached the predetermined number of times of setting or not (ST7).

If not reaching the number of times of setting, the measuring area is moved within the same well 7a (ST8).

The mechanism controller 14 controls the table moving mechanism 11. The micro plate 7 is moved relatively to the camera 13, and other measuring area set in the well 7a is moved into the imaging field of the camera 13. Again, the condition pass/fail determining process at ST3 and after is repeated.

If reaching the number of times of setting at ST7, it is judged whether or not to change the condition (ST9).

If the condition can be changed, the process for relaxing the condition is carried out (ST10). That is, by increasing the value of B showing the allowable range width at ST5, the allowable range of the number of cells N is expanded, and the process returns to ST2 and the subsequent process is repeated.

If the condition cannot be changed, presence or absence of any well to be measured which is not measured yet is judged (ST11).

If there is a well to be measured which is not measured yet, a well to be measured is changed to a well not measured yet (ST12).

Returning back to ST2, the subsequent process is repeated. At ST11, if there is no well to be measured which is not measured yet, the measuring process is terminated.

Referring now to FIG. 5, digitizing process on the image judged to conform to the condition is explained.

First, in the image to be measured, length L and area S1 of axon of cell are measured as linear structure numerical indices (ST21).

This process is done, as shown in FIG. 6C. In this process, the length and area of the linear structures 30b corresponding to the axon of cell is predetermined by image processing calculation on the processed image including only the linear structures 30b acquired from the original image shown in FIG. 6A.

Then, on the processed image shown in FIG. 6B, by determining the area of the main bodies 30a similarly by image processing calculation, the soma area S2 is measured as the soma numerical index (ST22).

Thus obtained numerical data N, L, S1, S2 are stored in the numerical data memory 22 (ST23).

On the basis of these numerical data, d1 (L)/(N), d2=(S1)/(N), d3=(L)/(S2), and d4=(S1)/(S2) are calculated (ST24).

The calculated numerical indices d1, d2, d3, d4 are stored in the numerical data memory 22 (ST25).

These numerical indices show the quantity of linear structures per a soma. At this time, the position information of the measuring area to be measured and the image data of the measuring area are also stored in relation to these numerical indices.

Then, adding 1 to the value of digitization process counter J, J=J+1 is set (ST26).

It is judged whether this counter value J have reached the predetermined number of times of measurement or not (ST27).

If not reaching the number of times of measurement, the measuring area is moved within the same well 7a (ST8).

That is, the mechanism controller 14 controls the table moving mechanism 11, and the micro plate 7 is moved relatively to the camera 13. Further, the mechanism controller 14 moves other measuring area in the well 7a into the imaging field of the camera 13. The condition pass/fail determining process at ST3 and after are repeated.

If reaching the number of times of measurement at ST27, the average of the numerical data in the well 7a is calculated (ST28).

Herein, D1=(sum of d1)/J, D2=(sum of d2)/J, D3=(sum of d3)/J, and D4=(sum of d4)/J are calculated. The determined D1, D2, D3, D4 are stored in the numerical data memory (ST29).

Then, returning back to ST11 shown in FIG. 4, the same process is repeated.

As explained above, the evaluation method of biological samples is a method for evaluating biological samples by acquiring numerical data such as length of neurite showing the degree of growth of neurite from the image data obtained by taking an image of the state of the soma of the biological sample. Herein, the biological sample, that is, the soma is stored in the micro plate 7, which is used as a container.

In this method, from the measuring area set in the image to be evaluated, the condition for acquiring numerical data adequately, that is, the condition in which the number of cells existing in the measuring area to be in appropriate range is predetermined as the condition to be satisfied.

In acquiring numerical data, whether or not the measuring area conforms to this condition is determined by the condition pass/fail determining unit. If judged to conform to the condition, the numerical data is obtained from this measuring area. If judged not to conform to the condition, the measuring area is changed.

As a result, when measuring the degree of growth of neurite, the range of excessively concentrated proliferation area of cells or the range of an extremely small number of cells is excluded. Therefore, numerical data is acquired only from appropriate measuring area. It is hence free from fluctuation in precision or reliability of data depending on experience or skill of individual workers, so that highly objective numerical data can be acquired efficiently.

In the embodiment, having the camera 13 for taking an image of the micro plate 7, the camera 13 is moved relatively to the micro plate, and while acquiring the image data, the numerical data is acquired from the measuring area set in the image.

Or the obtained image data may be once stored in the first image memory 16, and by reading out the image data sequentially, the measuring area is set in the image. In this case, by changing the setting position of the measuring area set in the image showing one well, the measuring area is changed.

Herein, the reading controller for controlling the range of the image data being read out from the first image memory 16 functions as the measuring area changing unit.

In this embodiment, as the condition to be satisfied, an appropriate range of the number of cells N is predetermined.

The condition in the invention is not limited to such a condition determined on the basis of the numerical value.

This condition may be determined by preparing the reference image to be compared with the image to be evaluated as the condition. For example, the image in the measuring area and reference image are compared by pattern matching or the like, and the specified range of the obtained matching rate (result of comparison) may be defined as the reference for condition pass/fail determination.

In this method, various conditions may be set.

As the condition, aside from the number of cells, various conditions may be applied, or may be set depending on the purpose of observation.

According to the invention, the condition for acquiring numerical data appropriately from the image to be evaluated is predetermined as the condition to be satisfied. When acquiring the numerical data, the image to be evaluated is judged whether or not to conform to the condition by the condition pass/fail determining unit. Numerical data is obtained from the image judged to conform to the condition. Thus, only from the appropriate object, highly objective numerical data can be acquired efficiently.

What is claimed is:

1. An evaluation apparatus for a biological sample for acquiring numerical data showing a state of said biological sample from image data obtained by taking an image of said biological sample comprising:
(a) a condition pass/fail determining unit for determining whether a measuring area being set as a numerical data acquiring area in an image to be evaluated conforms to a predetermined condition for acquiring the numerical data,
(b) a digitization unit for acquiring the numerical data from the image of which the measuring area is judged to be conforming to the predetermined condition, and
(c) a measuring area changing unit for changing the measuring area.

2. The evaluation apparatus of claim 1, wherein the biological sample includes a cell, and the predetermined condition includes at least one of a number of the cell and an area of the cell in the measuring area.

3. The evaluation apparatus of claim 1, wherein the predetermined condition includes a reference image to be compared with the image in the measuring area.

4. The evaluation apparatus of claim 1, wherein the predetermined condition includes a predetermined value relating to a result of comparison obtained by comparison between the image in the measuring area and a reference image.

5. The evaluation apparatus of claim 1, wherein said biological sample is a cell having a linear structure extending from a soma, and said numerical data includes at least one of a length and an area of the linear structure.

6. An evaluation method for a biological sample for acquiring numerical data from an image data obtained by taking an image of said biological sample comprising the steps of:
(a) setting a condition for acquiring numerical data from a measuring area being set as a numerical data acquiring area in an image to be evaluated,
(b) judging whether the measuring area conforms to the condition when acquiring the numerical data,
(c) acquiring the numerical data from the measuring area when judged to conform to the condition, and
(d) changing the measuring area when judged not to conform.

7. The evaluation method of claim 6, wherein the biological sample includes a cell, and the condition includes at least one of a number of the cell and an area of the cell in the measuring area.

8. The evaluation method of claim 6, wherein the condition includes a reference image to be compared with the image in the measuring area.

9. The evaluation method of claim 6, wherein the predetermined condition includes a predetermined value relating to a result of comparison obtained by comparison between the image in the measuring area and a reference image.

10. The evaluation method of claim 6, wherein said biological sample is a cell having a linear structure extending from a soma, and said numerical data includes at least one of a length and an area of the linear structure.

11. A storage medium storing a computer program for executing an evaluation method of a biological sample for acquiring numerical data from an image data obtained by taking an image of said biological sample, said evaluation method comprising the steps of:
(a) setting a condition for acquiring numerical data from a measuring area being set as a numerical data acquiring area in an image to be evaluated,
(b) judging whether the measuring area conforms to the condition when acquiring the numerical data,
(c) acquiring the numerical data from the measuring area when judged to conform to the condition, and
(d) changing the measuring area when judged not to conform.

12. The storage medium of claim 11, wherein the biological sample include a cell, and the condition includes at least one of a number of cell and an area of the cell in the measuring area.

13. The storage medium of claim 11, wherein the condition includes a reference image to be compared with the image in the measuring area.

14. The storage medium of claim 11, wherein the condition includes the predetermined value relating to the result of comparison obtained by comparison between the image in the measuring area and a reference image.

15. The storage medium of claim 11, wherein said biological sample are a cell having a linear structure extending from the soma, and the numerical data includes at least one of a length and an area of the linear structure.
